(19) United States
(12) Patent Application Publication
(10) Pub. No.: US 2024/0216315 A1
(43) Pub. Date: Jul. 4, 2024

(54) METHODS AND COMPOSITIONS FOR TREATMENT OF DIABETIC RETINOPATHY AND RELATED CONDITIONS

(71) Applicant: Ocuphire Pharma, Inc., Farmington Hills, MI (US)

(72) Inventors: Mina Sooch, Bloomfield, MI (US); Konstantinos Charizanis, Ypsilanti, MI (US); Mark R. Kelley, Zionsville, IN (US); Richard Adam Messmann, Brighton, MI (US); Mitchell George Brigell, Belmont, MA (US); Ronil Ajaykumar Patel, Tampa, FL (US)

(21) Appl. No.: 18/288,435

(22) PCT Filed: Apr. 29, 2022
(86) PCT No.: PCT/US2022/027062

§ 371 Date: Oct. 26, 2023

Related U.S. Application Data
(60) Provisional application No. 63/182,037, filed on Apr. 30, 2021.

(51) Int. Cl. A61K 31/20; A61K 45/06; A61P 27/02

(52) U.S. Cl. CPC A61K 31/20; A61K 45/06; A61P 27/02

(57) ABSTRACT

The invention provides methods, compositions, and kits containing a first therapeutic agent that is a substituted 2,3-dimethoxyquinone of Formula I, or a pharmaceutically acceptable salt thereof, for treating patients suffering from diabetic retinopathy, diabetic macular edema, and/or other diabetic retinal disorders and/or other disorders.

### CROSS-REFERENCE TO RELATED APPLICATIONS

This application is the national stage application of International (PCT) Patent Application Serial No. PCT/US2022/027062, filed Apr. 29, 2022, which claims the benefit of and priority to U.S. Provisional Patent Application Ser. No. 63/182,037, filed Apr. 30, 2021; the contents of which are hereby incorporated by reference in their entirety.

### FIELD OF THE INVENTION

The invention provides methods, compositions, and kits containing a first therapeutic agent that is a substituted 2,3-dimethoxyquinone of Formula I, or a pharmaceutically acceptable salt thereof, for treating patients suffering from diabetic retinopathy, diabetic macular edema, and/or other diabetic retinal disorders and/or other disorders.

### BACKGROUND

Diabetic retinopathy is a disease of the eye that, if left untreated, can lead to blindness. A significant proportion of individuals who suffer from diabetes experience some degree of related retinal damage. Existing therapies for diabetic retinopathy are not effective for all patients and/or have undesirable side effects. For example, laser photocoagulation produces its effects by creating burns in the tissue of the eye, which can be painful and/or cause certain vision problems (e.g., losses in peripheral, color, and/or night vision). Vitrectomy generally proceeds by creating an incision in the surface of the eye (introducing the potential for intraocular infection), and often requires weeks of recovery where the eye must be covered and cannot be used. Intravitreal injection of triamcinolone or anti-VEGF medications also carry a risk of intraocular infection, particularly with the need for additional injections over time.

The compound (E)-2-((4,5-dimethoxy-2-methyl-3,6-dioxocyclohexa-1,4-dien-1-yl)methylene)undecanoic acid, which has the following chemical formula, is described in WO 2009/042542:

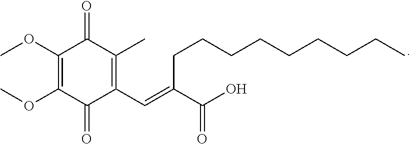 [SMILES: CCCCCCCCC/C(=C\C1=C(C)C(=O)C(OC)=C(OC)C1=O)C(=O)O]

Improved dosing procedures for treating diabetic retinopathy, diabetic macular edema, and/or other diabetic retinal disorders and/or other disorders using the foregoing compound would benefit patients.

The present invention addresses this need and provides other related advantages.

### SUMMARY

The invention provides methods, compositions, and kits containing a first therapeutic agent that is a substituted 2,3-dimethoxyquinone of Formula I, or a pharmaceutically acceptable salt thereof, for treating patients suffering from diabetic retinopathy, diabetic macular edema, and/or other diabetic retinal disorders and/or other disorders. The methods generally comprise orally administering to a human patient in need thereof an amount of from about 120 mg to about 600 mg per day of a compound of Formula I or a pharmaceutically acceptable salt thereof:

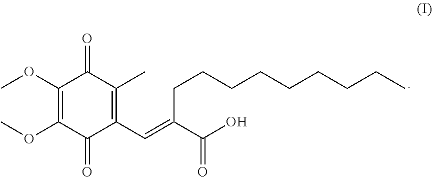 [SMILES: CCCCCCCCC/C(=C\C1=C(C)C(=O)C(OC)=C(OC)C1=O)C(=O)O]

Exemplary more preferred embodiments comprise orally administering to a human patient in need thereof an amount of from about 480 mg to about 600 mg per day of a compound of Formula I or pharmaceutically acceptable salt thereof. Improvement in the patient's diabetic retinal disorder can be evaluated according to improvement in the patient's Diabetic Retinopathy Severity Score (DRSS), improvement in the patient's visual acuity, and other procedures described in the literature. Additional exemplary aspects and embodiments of the invention are described below.

One aspect of the invention provides a method of treating a diabetic retinal disease in a human patient. The method comprises orally administering to a human patient in need thereof a first therapeutic agent in an amount of from about 480 mg to about 600 mg per day, to thereby treat the diabetic retinal disease, wherein the first therapeutic agent is a compound of Formula I or a pharmaceutically acceptable salt thereof:

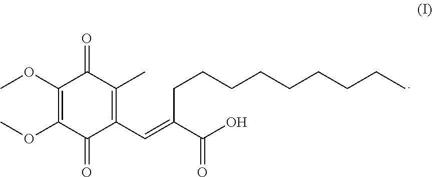 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the method further comprises administering to the patient a second therapeutic agent that is a vascular endothelial growth factor inhibitor. In certain embodiments, the diabetic retinal disease is diabetic retinopathy. In certain embodiments, the diabetic retinal disease is diabetic macular edema. Additional features of the method are described in the detailed description.

Another aspect of the invention provides a method of treating a diabetic retinal disease in a human patient. The method comprises orally administering to a human patient in need thereof a first therapeutic agent in an amount of from about 120 mg to about 600 mg per day, to thereby treat the diabetic retinal disease, wherein the first therapeutic agent is a compound of Formula I or a pharmaceutically acceptable salt thereof:

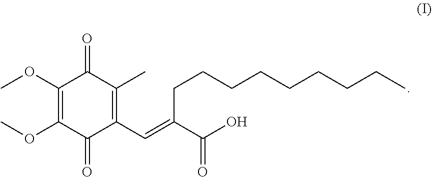 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the method further comprises administering to the patient a second therapeutic agent that is a vascular endothelial growth factor inhibitor. In certain embodiments, the diabetic retinal disease is diabetic retinopathy. In certain embodiments, the diabetic retinal disease is diabetic macular edema. Additional features of the method are described in the detailed description.

Another aspect of the invention provides a pharmaceutical composition comprising a compound of Formula I or a pharmaceutically acceptable salt thereof, for use in treating a diabetic retinal disease in a human patient according to a method described herein. Preferably, the pharmaceutical composition is formulated for oral administration.

Another aspect of the invention provides a method of treating a disease or condition selected from wet age-related macular degeneration, dry age-related macular degeneration, retinal vein occlusion, geographic atrophy, retinal neovascularization, choroidal neovascularization, or corneal graft rejection. The method comprises orally administering to a human patient in need thereof a first therapeutic agent in an amount of from about 120 mg to about 600 mg per day, to thereby treat the disease or condition, wherein the first therapeutic agent is a compound of Formula I or a pharmaceutically acceptable salt thereof:

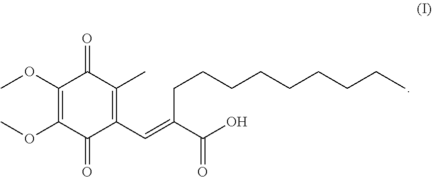 [SMILES: CCCCCCCCC/C(=C\C1=C(C)C(=O)C(OC)=C(OC)C1=O)C(=O)O]

### BRIEF DESCRIPTION OF FIGURES

FIG. 1 depicts exemplary diabetic retinopathy severity scores (DRSS) and corresponding descriptions and retinal images.

### DETAILED DESCRIPTION OF THE INVENTION

The invention provides methods, compositions, and kits containing a first therapeutic agent that is a substituted 2,3-dimethoxyquinone of Formula I, or a pharmaceutically acceptable salt thereof, for treating patients suffering from diabetic retinopathy, diabetic macular edema, and/or other diabetic retinal disorders and/or other disorders. The methods generally comprise orally administering to a human patient in need thereof an amount of from about 120 mg to about 600 mg per day of a compound of Formula I or a pharmaceutically acceptable salt thereof:

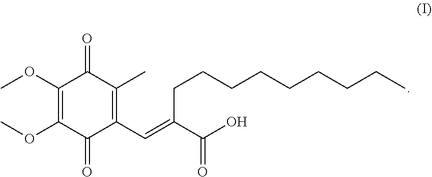 [SMILES: CCCCCCCCC/C(=C\C1=C(C)C(=O)C(OC)=C(OC)C1=O)C(=O)O]

Exemplary more preferred embodiments comprise orally administering to a human patient in need thereof an amount of from about 480 mg to about 600 mg per day of a compound of Formula I or pharmaceutically acceptable salt thereof. Improvement in the patient's diabetic retinal disorder can be evaluated according to improvement in the patient's Diabetic Retinopathy Severity Score (DRSS), improvement in the patient's visual acuity, and other procedures described in the literature. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate.

The term “about” means within 10% of the stated value. In certain embodiments, the value may be within 8%, 6%, 5%, 4%, 2%, or 1% of the stated value.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating patients suffering from diabetic retinopathy, diabetic macular edema, and/or other diabetic retinal disorders by orally administering to a human patient a substituted 2,3-dimethoxyquinone of Formula I, or a pharmaceutically acceptable salt thereof. The invention also provides methods for treating patients suffering from other disorders by administering to a human patient a substituted 2,3-dimethoxyquinone of Formula I, or a pharmaceutically acceptable salt thereof. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating a diabetic retinal disease in a human patient, comprising orally administering to a human patient in need thereof a first therapeutic agent in an amount of from about 480 mg to about 600 mg per day, to thereby treat the diabetic retinal disease, wherein the first therapeutic agent is a compound of Formula I or a pharmaceutically acceptable salt thereof:

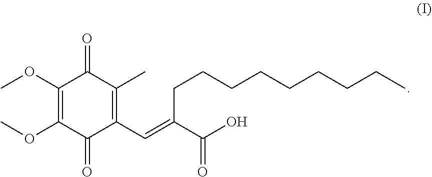 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in an amount of about 600 mg per day.

In certain embodiments, about 360 mg of the first therapeutic agent is orally administered to the patient in the morning, and about 240 mg of the first therapeutic agent is orally administered to the patient in the evening. In certain embodiments, about 360 mg of the first therapeutic agent is orally administered to the patient, and then at a time that is from about 8 hours to about 16 hours later about 240 mg of the first therapeutic agent is orally administered to the patient. In certain embodiments, about 360 mg of the first therapeutic agent is orally administered to the patient, and then at a time that is from about 10 hours to about 14 hours later about 240 mg of the first therapeutic agent is orally administered to the patient.

In certain embodiments, about 240 mg of the first therapeutic agent is orally administered to the patient in the morning, and about 360 mg of the first therapeutic agent is orally administered to the patient in the evening. In certain embodiments, about 240 mg of the first therapeutic agent is orally administered to the patient, and then at a time that is from about 8 hours to about 16 hours later about 360 mg of the first therapeutic agent is orally administered to the patient. In certain embodiments, about 240 mg of the first therapeutic agent is orally administered to the patient, and then at a time that is from about 10 hours to about 14 hours later about 360 mg of the first therapeutic agent is orally administered to the patient.

In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient, and then at a time that is from about 10 hours to about 14 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 480 mg per day.

In certain embodiments, the first therapeutic agent is orally administered to a patient in an amount of about 480 mg per day.

In certain embodiments, about 240 mg of the first therapeutic agent is orally administered to the patient in the morning, and about 240 mg of the first therapeutic agent is orally administered to the patient in the evening. In certain embodiments, about 240 mg of the first therapeutic agent is orally administered to the patient, and then at a time that is from about 8 hours to about 16 hours later about 240 mg of the first therapeutic agent is orally administered to the patient. In certain embodiments, about 240 mg of the first therapeutic agent is orally administered to the patient, and then at a time that is from about 10 hours to about 14 hours later about 240 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 300 mg per day. In certain embodiments, the first therapeutic agent is orally administered to the patient in the morning.

### B. Second Method

Another aspect of the invention provides a method of treating a diabetic retinal disease in a human patient, comprising orally administering to a human patient in need thereof a first therapeutic agent in an amount of from about 120 mg to about 600 mg per day, to thereby treat the diabetic retinal disease, wherein the first therapeutic agent is a compound of Formula I or a pharmaceutically acceptable salt thereof:

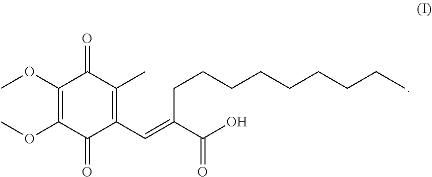 [SMILES: CCCCCCCCC/C(=C\C1=C(C)C(=O)C(OC)=C(OC)C1=O)C(=O)O]

The method may be further characterized by additional features, such as the identity of the first therapeutic agent, the dosing amount, and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing amount. For example, in certain embodiments, the first therapeutic agent is orally administered to the patient in an amount of about 300 mg per day. In certain embodiments, the first therapeutic agent is orally administered to the patient in an amount of about 240 mg per day. In certain embodiments, the first therapeutic agent is orally administered to the patient in an amount of about 120 mg per day.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in the morning. In certain embodiments, the first therapeutic agent is orally administered to the patient in the evening.

### C. Third Method

One aspect of the invention provides a method of reducing angiogenesis in retinal tissue in a human patient suffering from a diabetic retinal disease, comprising orally administering to a human patient in need thereof a first therapeutic agent in an amount of from about 480 mg to about 600 mg per day, to reduce angiogenesis in retinal tissue, wherein the first therapeutic agent is a compound of Formula I or a pharmaceutically acceptable salt thereof:

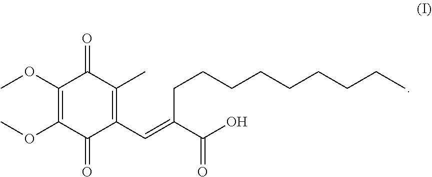 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in an amount of about 600 mg per day.

In certain embodiments, about 360 mg of the first therapeutic agent is orally administered to the patient in the morning, and about 240 mg of the first therapeutic agent is orally administered to the patient in the evening. In certain embodiments, about 360 mg of the first therapeutic agent is orally administered to the patient, and then at a time that is from about 8 hours to about 16 hours later about 240 mg of the first therapeutic agent is orally administered to the patient. In certain embodiments, about 360 mg of the first therapeutic agent is orally administered to the patient, and then at a time that is from about 10 hours to about 14 hours later about 240 mg of the first therapeutic agent is orally administered to the patient.

In certain embodiments, about 240 mg of the first therapeutic agent is orally administered to the patient in the morning, and about 360 mg of the first therapeutic agent is orally administered to the patient in the evening. In certain embodiments, about 240 mg of the first therapeutic agent is orally administered to the patient, and then at a time that is from about 8 hours to about 16 hours later about 360 mg of the first therapeutic agent is orally administered to the patient. In certain embodiments, about 240 mg of the first therapeutic agent is orally administered to the patient, and then at a time that is from about 10 hours to about 14 hours later about 360 mg of the first therapeutic agent is orally administered to the patient.

In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient, and then at a time that is from about 10 hours to about 14 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 480 mg per day.

In certain embodiments, the first therapeutic agent is orally administered to a patient in an amount of about 480 mg per day.

In certain embodiments, about 240 mg of the first therapeutic agent is orally administered to the patient in the morning, and about 240 mg of the first therapeutic agent is orally administered to the patient in the evening. In certain embodiments, about 240 mg of the first therapeutic agent is orally administered to the patient, and then at a time that is from about 8 hours to about 16 hours later about 240 mg of the first therapeutic agent is orally administered to the patient. In certain embodiments, about 240 mg of the first therapeutic agent is orally administered to the patient, and then at a time that is from about 10 hours to about 14 hours later about 240 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 300 mg per day. In certain embodiments, the first therapeutic agent is orally administered to the patient in the morning.

### D. Fourth Method

Another aspect of the invention provides a method of reducing angiogenesis in retinal tissue in a human patient suffering from a diabetic retinal disease, comprising orally administering to a human patient in need thereof a first therapeutic agent in an amount of from about 120 mg to about 600 mg per day, to reduce angiogenesis in retinal tissue, wherein the first therapeutic agent is a compound of Formula I or a pharmaceutically acceptable salt thereof:

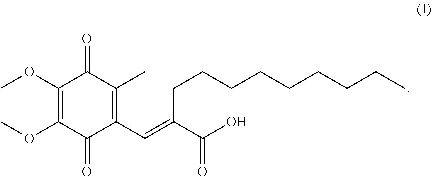 [SMILES: CCCCCCCCC/C(=C\C1=C(C)C(=O)C(OC)=C(OC)C1=O)C(=O)O]

The method may be further characterized by additional features, such as the identity of the first therapeutic agent, the dosing amount, and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing amount. For example, in certain embodiments, the first therapeutic agent is orally administered to the patient in an amount of about 300 mg per day. In certain embodiments, the first therapeutic agent is orally administered to the patient in an amount of about 240 mg per day. In certain embodiments, the first therapeutic agent is orally administered to the patient in an amount of about 120 mg per day.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in the morning. In certain embodiments, the first therapeutic agent is orally administered to the patient in the evening.

### E. Fifth Method

One aspect of the invention provides a method of reducing the activity of HIF-1α and/or NF-κB in a human patient suffering from a diabetic retinal disease, comprising orally administering to a human patient in need thereof a first therapeutic agent in an amount of from about 480 mg to about 600 mg per day, to reduce the activity of HIF-1α and/or NF-κB, wherein the first therapeutic agent is a compound of Formula I or a pharmaceutically acceptable salt thereof:

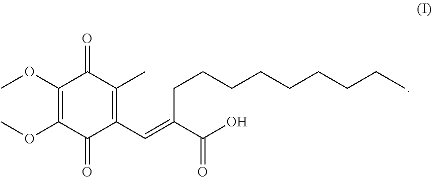 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in an amount of about 600 mg per day.

In certain embodiments, about 360 mg of the first therapeutic agent is orally administered to the patient in the morning, and about 240 mg of the first therapeutic agent is orally administered to the patient in the evening. In certain embodiments, about 360 mg of the first therapeutic agent is orally administered to the patient, and then at a time that is from about 8 hours to about 16 hours later about 240 mg of the first therapeutic agent is orally administered to the patient. In certain embodiments, about 360 mg of the first therapeutic agent is orally administered to the patient, and then at a time that is from about 10 hours to about 14 hours later about 240 mg of the first therapeutic agent is orally administered to the patient.

In certain embodiments, about 240 mg of the first therapeutic agent is orally administered to the patient in the morning, and about 360 mg of the first therapeutic agent is orally administered to the patient in the evening. In certain embodiments, about 240 mg of the first therapeutic agent is orally administered to the patient, and then at a time that is from about 8 hours to about 16 hours later about 360 mg of the first therapeutic agent is orally administered to the patient. In certain embodiments, about 240 mg of the first therapeutic agent is orally administered to the patient, and then at a time that is from about 10 hours to about 14 hours later about 360 mg of the first therapeutic agent is orally administered to the patient.

In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient, and then at a time that is from about 10 hours to about 14 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 480 mg per day.

In certain embodiments, the first therapeutic agent is orally administered to a patient in an amount of about 480 mg per day.

In certain embodiments, about 240 mg of the first therapeutic agent is orally administered to the patient in the morning, and about 240 mg of the first therapeutic agent is orally administered to the patient in the evening. In certain embodiments, about 240 mg of the first therapeutic agent is orally administered to the patient, and then at a time that is from about 8 hours to about 16 hours later about 240 mg of the first therapeutic agent is orally administered to the patient. In certain embodiments, about 240 mg of the first therapeutic agent is orally administered to the patient, and then at a time that is from about 10 hours to about 14 hours later about 240 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 300 mg per day. In certain embodiments, the first therapeutic agent is orally administered to the patient in the morning.

### F. Sixth Method

One aspect of the invention provides a method of reducing the activity of HIF-1α and/or NF-κB in a human patient suffering from a diabetic retinal disease, comprising orally administering to a human patient in need thereof a first therapeutic agent in an amount of from about 120 mg to about 600 mg per day, to reduce the activity of HIF-1α and/or NF-κB, wherein the first therapeutic agent is a compound of Formula I or a pharmaceutically acceptable salt thereof:

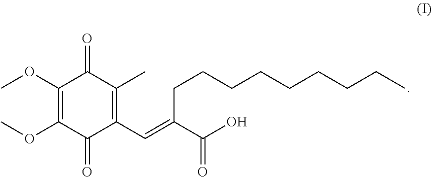 [SMILES: CCCCCCCCC/C(=C\C1=C(C)C(=O)C(OC)=C(OC)C1=O)C(=O)O]

The method may be further characterized by additional features, such as the identity of the first therapeutic agent, the dosing amount, and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing amount. For example, in certain embodiments, the first therapeutic agent is orally administered to the patient in an amount of about 300 mg per day. In certain embodiments, the first therapeutic agent is orally administered to the patient in an amount of about 240 mg per day. In certain embodiments, the first therapeutic agent is orally administered to the patient in an amount of about 120 mg per day.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in the morning. In certain embodiments, the first therapeutic agent is orally administered to the patient in the evening.

### G. Seventh Method

One aspect of the invention provides a method of treating a disease or condition in a human patient, comprising administering to a human patient in need thereof a first therapeutic agent to thereby treat the disease or condition, wherein the first therapeutic agent is a compound of Formula I or a pharmaceutically acceptable salt thereof:

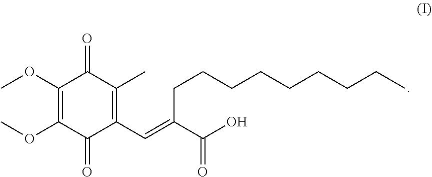 [SMILES: CCCCCCCCC/C(=C\C1=C(C)C(=O)C(OC)=C(OC)C1=O)C(=O)O]

The method may be further characterized by additional features, such as the identity of the disease or condition, the identity of the first therapeutic agent, the dosing amount, and the dosing regimen. The invention embraces all permutations and combinations of these features.

Accordingly, the method may be further characterized according to the identity the disease or condition. In certain embodiments, the disease or condition is selected from wet age-related macular degeneration, dry age-related macular degeneration, retinal vein occlusion, geographic atrophy, retinal neovascularization, choroidal neovascularization, or corneal graft rejection. In certain embodiments, the disease or condition is wet age-related macular degeneration. In certain embodiments, the disease or condition is dry age-related macular degeneration. In certain embodiments, the disease or condition is retinal vein occlusion. In certain embodiments, the disease or condition is geographic atrophy. In certain embodiments, the disease or condition is retinal neovascularization. In certain embodiments, the disease or condition is choroidal neovascularization. In certain embodiments, the disease or condition is corneal graft rejection.

In certain embodiments, the disease or condition is ocular oncology. In certain embodiments, the disease or condition is a solid tumor. In certain embodiments, the disease or condition is a cancer due to human myeloid leukemia mononuclear cell line (THP-1). In certain embodiments, the disease or condition is Barrett's esophagus (BE). In certain embodiments, the disease or condition is metaplastic Barrett's esophagus (BE). In certain embodiments, the disease or condition is an esophageal adenocarcinoma.

In certain embodiments, the disease or condition is dry eye disease, uveitis, liver disease (e.g., hepatitis, NASH, or alcoholic steatosis), thyroid eye disease, sickle cell retinopathy, chemotherapy-induced peripheral neuropathy, irritable bowel syndrome, stroke, gastro-intestinal disfunction, or chronic gastroesophageal reflux disease (GERD). In certain embodiments, the disease or condition is an inflammatory skin disorder. In certain embodiments, the disease or condition is psoriasis, atopic dermatitis, or rosacea. In certain embodiments, the disease or condition is dry eye disease. In certain embodiments, the disease or condition is uveitis. In certain embodiments, the disease or condition is liver disease (e.g., hepatitis, NASH, or alcoholic steatosis). In certain embodiments, the disease or condition is thyroid eye disease. In certain embodiments, the disease or condition is inherited retinal diseases (e.g., retinitis pigmentosa, choroideremia, Stargardt disease, cone-rod dystrophy, or Leber Congenital Amaurosis). In certain embodiments, the disease or condition is sickle cell retinopathy. In certain embodiments, the disease or condition is chemotherapy-induced peripheral neuropathy. In certain embodiments, the disease or condition is irritable bowel syndrome. In certain embodiments, the disease or condition is stroke. In certain embodiments, the disease or condition is gastro-intestinal disfunction. In certain embodiments, the disease or condition is chronic gastroesophageal reflux disease (GERD).

In certain embodiments, the disease or condition is diabetic retinal disease.

As indicated above, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized according to the dosing amount. For example, in certain embodiments, the first therapeutic agent is orally administered to the patient in an amount of from about 120 mg to about 600 mg per day. In certain embodiments, the first therapeutic agent is orally administered to the patient in an amount of from about 480 mg to about 600 mg per day. In certain embodiments, the first therapeutic agent is orally administered to the patient in an amount of about 600 mg per day. In certain embodiments, the first therapeutic agent is orally administered to the patient in an amount of about 480 mg per day. In certain embodiments, the first therapeutic agent is orally administered to the patient in an amount of about 300 mg per day. In certain embodiments, the first therapeutic agent is orally administered to the patient in an amount of about 240 mg per day. In certain embodiments, the first therapeutic agent is orally administered to the patient in an amount of about 120 mg per day.

The method may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in the morning. In certain embodiments, the first therapeutic agent is orally administered to the patient in the evening.

In certain embodiments, about 360 mg of the first therapeutic agent is orally administered to the patient in the morning, and about 240 mg of the first therapeutic agent is orally administered to the patient in the evening. In certain embodiments, about 360 mg of the first therapeutic agent is orally administered to the patient, and then at a time that is from about 8 hours to about 16 hours later about 240 mg of the first therapeutic agent is orally administered to the patient. In certain embodiments, about 360 mg of the first therapeutic agent is orally administered to the patient, and then at a time that is from about 10 hours to about 14 hours later about 240 mg of the first therapeutic agent is orally administered to the patient.

In certain embodiments, about 240 mg of the first therapeutic agent is orally administered to the patient in the morning, and about 360 mg of the first therapeutic agent is orally administered to the patient in the evening. In certain embodiments, about 240 mg of the first therapeutic agent is orally administered to the patient, and then at a time that is from about 8 hours to about 16 hours later about 360 mg of the first therapeutic agent is orally administered to the patient. In certain embodiments, about 240 mg of the first therapeutic agent is orally administered to the patient, and then at a time that is from about 10 hours to about 14 hours later about 360 mg of the first therapeutic agent is orally administered to the patient.

In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient, and then at a time that is from about 10 hours to about 14 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 480 mg per day.

In certain embodiments, about 240 mg of the first therapeutic agent is orally administered to the patient in the morning, and about 240 mg of the first therapeutic agent is orally administered to the patient in the evening. In certain embodiments, about 240 mg of the first therapeutic agent is orally administered to the patient, and then at a time that is from about 8 hours to about 16 hours later about 240 mg of the first therapeutic agent is orally administered to the patient. In certain embodiments, about 240 mg of the first therapeutic agent is orally administered to the patient, and then at a time that is from about 10 hours to about 14 hours later about 240 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 300 mg per day. In certain embodiments, the first therapeutic agent is orally administered to the patient in the morning.

Combinations of the embodiments recited herein above are part of the invention. For example, in certain embodiments, the invention provides a method of treating a disease or condition selected from wet age-related macular degeneration, dry age-related macular degeneration, retinal vein occlusion, geographic atrophy, retinal neovascularization, choroidal neovascularization, or corneal graft rejection, wherein the comprises orally administering to a human patient in need thereof a first therapeutic agent in an amount of from about 120 mg to about 600 mg per day, to thereby treat the disease or disease or condition, wherein the first therapeutic agent is a compound of Formula I or a pharmaceutically acceptable salt thereof:

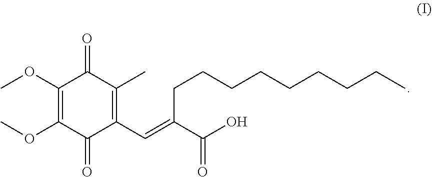 [SMILES: CCCCCCCCC/C(=C\C1=C(C)C(=O)C(OC)=C(OC)C1=O)C(=O)O]

### H. General Considerations for Therapeutic Methods

General considerations that may be applied to therapeutic methods described herein (e.g., the methods described in Parts A and G above) are provided below and include, for example, the duration of daily oral administration of the first therapeutic agent, characteristics of the disease or condition to be treated (e.g., characteristics of the diabetic retinal disease), and the identity of the human patient. A more thorough description of such features is provided below. The invention embraces all permutations and combinations of these features.

### Duration of Daily Administration of the First Therapeutic Agent

The methods may be further characterized according to the duration of daily oral administration of the first therapeutic agent. For example, in certain embodiments, the amount of the first therapeutic agent is orally administered to the patient daily for at least 1 week. In certain embodiments, the amount of the first therapeutic agent is orally administered to the patient daily for at least 2 weeks. In certain embodiments, the amount of the first therapeutic agent is orally administered to the patient daily for at least 4 weeks. In certain embodiments, the amount of the first therapeutic agent is orally administered to the patient daily for at least 6 weeks. In certain embodiments, the amount of the first therapeutic agent is orally administered to the patient daily for at least 8 weeks. In certain embodiments, the amount of the first therapeutic agent is orally administered to the patient daily for at least 10 weeks. In certain embodiments, the amount of the first therapeutic agent is orally administered to the patient daily for at least 12 weeks. In certain embodiments, the amount of the first therapeutic agent is orally administered to the patient daily for at least 24 weeks. In certain embodiments, the amount of the first therapeutic agent is orally administered to the patient daily for at least 30, 32, 34, 36, 38 40, 42, 44, 46, 48, 50, or 52 weeks.

### Characteristics of the Diabetic Retinal Disease

The methods may be further characterized according to characteristics of the diabetic retinal disease. For example, in certain embodiments, the diabetic retinal disease is diabetic retinopathy. In certain embodiments, the diabetic retinopathy is mild diabetic retinopathy. In certain embodiments, the diabetic retinopathy is moderate diabetic retinopathy. In certain embodiments, the diabetic retinopathy is moderately severe to severe diabetic retinopathy. In certain embodiments, the diabetic retinopathy is non-proliferative diabetic retinopathy. In certain embodiments, the diabetic retinopathy is proliferative diabetic retinopathy.

In certain embodiments, the diabetic retinal disease is diabetic macular edema.

### Additional Considerations

The methods may be further characterized according to additional considerations, such as the form in which the first therapeutic agent is administered, identity of the human patient, and improvement in diabetic retinal disease achieved by the method.

For example, in certain embodiments, the first therapeutic agent is orally administered to the patient in the form of an extended-release pharmaceutical composition. in certain embodiments, the first therapeutic agent is orally administered to the patient in the form of an extended-release pharmaceutical composition that provides release of the first therapeutic agent for duration of at least 1, 2, 3, 4, 5, 6, 7, 8, 9, 10, 11, or 12 hours. In certain embodiments, the first therapeutic agent is orally administered to the patient in the form of an immediate-release pharmaceutical composition. Pharmaceutical compositions are described in further detail in Section III below.

In certain embodiments, the human patient is an adult human patient.

In certain embodiments, the method is further characterized according to the improvement in diabetic retinopathy severity score. For example, in certain embodiments, the patient experiences a reduction of at 5, 10, 15, 20, 25, 30, 35, or 40 points in the diabetic retinopathy severity score due to the method. In certain embodiments, the patient experiences at least a two-step reduction in diabetic retinopathy severity score due to the method. In certain embodiments, the patient experiences at least a three-step reduction in diabetic retinopathy severity score due to the method. In certain embodiments, the patient experiences at least a four-step reduction in diabetic retinopathy severity score due to the method.

In certain embodiments, the method is further characterized according to the improvement in best-corrected visual acuity. For example, in certain embodiments, the patient experiences an improvement of at least 5%, 10%, 15%, 20%, 25%, 30%, 40%, 50%, 60%, 70%, 80%, 90% or 100% in best-corrected visual acuity due to the method. In certain embodiments, the patient experiences an improvement of at least 2, 4, 6, 8, 10, 12, 14, 16, or 18 letters in best-corrected visual acuity due to the method. Best-corrected visual acuity can be measured according to methods known in the art, for example, with a Standard ETDRS illuminated chart (on wall or stand) at 4 m. Alternatively, best-corrected visual acuity can be measured using a Snellen chart.

In certain embodiments, the method is further characterized according to impact on a symptom of diabetes. In certain embodiments, the method reduces a symptom of diabetes. In certain embodiments, the method reduces any renal impairment experienced by the patient. In certain embodiments, the method reduces any renal impairment experienced by the patient by at least 5, 10, 12, 14, 16, 18, 20, 22, 24, 26, 28, 30, 32, 34, 36, 38, 40, 50, 60, 70, 80, or 90 percent. The said reduction in renal impairment is relative to that experienced by a comparable patient that has not received therapy according to the method using the first therapeutic agent.

In certain embodiments, the method achieves a neuroprotective effect.

In certain embodiments, the method is further characterized by the feature that any increase in blood plasma concentration of alanine aminotransferase due to the first therapeutic agent is no greater than 5%. In certain embodiments, the method is further characterized by the feature that any increase in blood plasma concentration of alanine aminotransferase due to the first therapeutic agent is no greater than 2, 3, 4, 5, 6, 7, 8, 9, 10, 11, 12, 13, 14, 15, 16, 17, 18, 19, or 15 percent.

In certain embodiments, the method is further characterized by the feature it results in a reduction in blood plasma concentration of alanine aminotransferase due to the first therapeutic agent. In certain embodiments, the reduction is at least 2, 3, 4, 5, 6, 7, 8, 9, 10, 11, 12, 13, 14, 15, 16, 17, 18, 19, or 15 percent.

In certain embodiments, the method is further characterized by the feature that any increase in blood plasma concentration of aspartate aminotransferase due to the first therapeutic agent is no greater than 5%. In certain embodiments, the method is further characterized by the feature that any increase in blood plasma concentration of aspartate aminotransferase due to the first therapeutic agent is no greater than 2, 3, 4, 5, 6, 7, 8, 9, 10, 11, 12, 13, 14, 15, 16, 17, 18, 19, or 15 percent.

In certain embodiments, the method is further characterized by the feature it results in a reduction in blood plasma concentration of aspartate aminotransferase due to the first therapeutic agent. In certain embodiments, the reduction is at least 2, 3, 4, 5, 6, 7, 8, 9, 10, 11, 12, 13, 14, 15, 16, 17, 18, 19, or 15 percent.

In certain embodiments, the method is further characterized by the feature that any reduction in glomerular filtration rate in the patient is no greater than 15%. In certain embodiments, the method is further characterized by the feature that any reduction in glomerular filtration rate in the patient is no greater than 2, 3, 4, 5, 6, 7, 8, 9, 10, 11, 12, 13, 14, 15, 16, 17, 18, 19, or 15 percent.

In certain embodiments, the method is further characterized by the feature that the incidence of any eye disorder due to the first therapeutic agent occurs no more frequently than one patient for every ten patients subjected to the same treatment. In certain embodiments, the method is further characterized by the feature that the incidence of any eye disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any eye disorder due to the first therapeutic agent occurs no more frequently than one patient for every twenty patients subjected to the same treatment. In certain embodiments, the method is further characterized by the feature that the incidence of any eye disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any gastrointestinal disorder due to the first therapeutic agent occurs no more frequently than one patient for every ten patients subjected to the same treatment. In certain embodiments, the method is further characterized by the feature that the incidence of any gastrointestinal disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any nervous system disorder due to the first therapeutic agent occurs no more frequently than one patient for every twenty patients subjected to the same treatment. In certain embodiments, the method is further characterized by the feature that the incidence of any nervous system disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

Another aspect of the invention provides for the use of the first therapeutic agent described herein in the manufacture of a medicament. In certain embodiments, the medicament is for treating a disorder described herein, for example, for treating diabetic retinopathy, diabetic macular edema, and/or other diabetic retinal disorders.

Another aspect of the invention provides for the use of the first therapeutic agent described herein for treating a medical disorder, such as a medical disorder described herein, for example, for treating diabetic retinopathy, diabetic macular edema, and/or other diabetic retinal disorders.

### II. Combination Therapy

Another aspect of the invention provides for combination therapy. The First, Second, Third, Fourth, Fifth, Sixth, and Seventh Therapeutic Methods described hereinabove may optionally further comprise administering one or more second therapeutic agents to the patient. For example, in certain embodiments, the method further comprises administering to the patient a second therapeutic agent for treating diabetic retinal disease.

In certain embodiments, the second therapeutic agent that is an anti-inflammatory agent, anti-angiogenic agent, tyrosine kinase inhibitor, angiopoietin-2 inhibitor, and/or vascular endothelial growth factor inhibitor. In certain embodiments, the second therapeutic agent is a vascular endothelial growth factor inhibitor. In certain embodiments, the vascular endothelial growth factor inhibitor is sorafenib, sunitinib, pazopanib, bevacizumab, ranibizumab, aflibercept, nilotinib, or dasatinib. In certain embodiments, the vascular endothelial growth factor inhibitor is a bispecific antibody. In certain embodiments, the anti-inflammatory agent is a corticosteroid. In certain embodiments, the second therapeutic agent is a VEGF inhibitor, mTor inhibitor, VEGFR2 phosphorylation agent, tyrosine kinase inhibitor, IGF-1R inhibitor, nicotinic acetylcholine receptor antagonist, selective inhibitor of glycation, corticosteroid, NSAID, flavonoid, TNF alpha inhibitor, PKC inhibitor, aldose reductase, PARP inhibitor, reactive oxygen species inhibitor, AT-I Receptor modulator, AT-II receptor modular, rho associated protein kinase inhibitor, protease inhibitor, nitric oxide synthase inhibitor, AGE inhibitor, or PPAR-gamma up-regulator.

In certain embodiments, the second therapeutic agent is an immunoncology therapy, a Car-t therapy, a Crispr therapy, a BTK modulator, a bcl-2 modulator, a stat-3 modulator, a KRAS modulator, a PD1 modulator, and/or a DNA repair agent. In certain embodiments, the second therapeutic agent is a bone marrow transplant or related transplant. In certain embodiments, the modulator is an inhibitor.

In certain embodiments, the method further comprises administering to the patient a second therapeutic agent that is an anti-inflammatory agent, anti-angiogenic agent, tyrosine kinase inhibitor, angiopoietin-2 inhibitor, and/or vascular endothelial growth factor inhibitor. In certain embodiments, the method further comprises administering to the patient a second therapeutic agent that is a vascular endothelial growth factor inhibitor. In certain embodiments, the vascular endothelial growth factor inhibitor is sorafenib, sunitinib, pazopanib, bevacizumab, ranibizumab, aflibercept, nilotinib, or dasatinib. In certain embodiments, the vascular endothelial growth factor inhibitor is a bispecific antibody. In certain embodiments, the anti-inflammatory agent is a corticosteroid.

In certain embodiments, the

In certain embodiments, the first therapeutic agent is the only therapeutic agent for treating diabetic retinal disease that is administered to the human patient.

In certain embodiments, such as when treating an inflammatory skin disease, the second therapeutic agent is an immunosuppressant, anti-inflammatory agent, light therapy (e.g., sunlight, UVA, UVB, Psoralen UVA, or Excimer laser), a retinoid, a corticosteroid, a Vitamin D analogue, a calcineurin inhibitor, salicylic acid, anthralin, coal tar, or Goeckerman therapy (e.g., light and coal tar).

The second therapeutic agent and optionally additional therapeutic agents may be administered separately from a compound or composition of the invention, as part of a multiple dosage regimen. Alternatively, the second therapeutic agent and optionally additional therapeutic agents may be part of a single dosage form, mixed together with a compound of this invention in a single composition. If administered as a multiple dosage regime, the second therapeutic agent and optionally additional therapeutic agents and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, the second therapeutic agent and optionally additional therapeutic agents and a compound or composition of the invention are administered as a multiple dosage regimen more than 24 hours apart.

### III. Pharmaceutical Compositions

As indicated above, the invention provides pharmaceutical compositions, which comprise a therapeutically effective amount of one or more of the compounds described above, formulated together with one or more pharmaceutically acceptable carriers (additives) and/or diluents. The pharmaceutical compositions may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

In certain embodiments, the pharmaceutical composition may be in the form of a cream, colloidal, suspension, spray, gel, lotion, ointment, foam, or solution. In certain embodiments, the pharmaceutical composition may be in the form of a solution for injection. In certain embodiments, the pharmaceutical composition may be in the form of a solution for sub-cutaneous injection.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a compound of the invention will be that amount of the compound which is the lowest dose effective to produce a therapeutic effect. Such an effective dose will generally depend upon the factors described above. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms.

The invention further provides a unit dosage form (such as a tablet or capsule) comprising a compound described herein in a therapeutically effective amount for the treatment of a medical disorder described herein.

### IV. Medical Kits

Another aspect of the invention provides a medical kit comprising, for example, (i) a therapeutic agent described herein, and (ii) instructions for treating diabetic retinopathy, diabetic macular edema, and/or other diabetic retinal disorders according to methods described herein.

### EXAMPLES

The invention now being generally described, will be more readily understood by reference to the following examples, which are included merely for purposes of illustrating certain aspects and embodiments of the present invention, and are not intended to limit the invention.

### Example 1—Treatment of Non-Proliferative Diabetic Retinopathy and Mild Proliferative Diabetic Retinopathy

Ability of Compound 1 to treat non-proliferative diabetic retinopathy (NPDR) and mild proliferative diabetic retinopathy (PDR) may be evaluated according to a clinical study in which Compound 1 is orally administered to patients suffering from non-proliferative diabetic retinopathy or mild proliferative diabetic retinopathy. Compound 1 has the chemical name (E)-2-((4,5-dimethoxy-2-methyl-3,6-dioxocyclohexa-1,4-dien-1-yl)methylene)undecanoic acid, and is depicted by the following chemical formula:

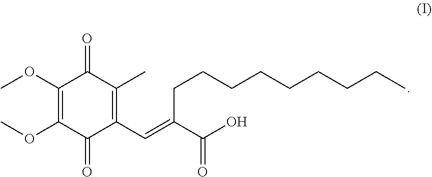 [SMILES: CCCCCCCCC/C(=C\C1=C(C)C(=O)C(OC)=C(OC)C1=O)C(=O)O]

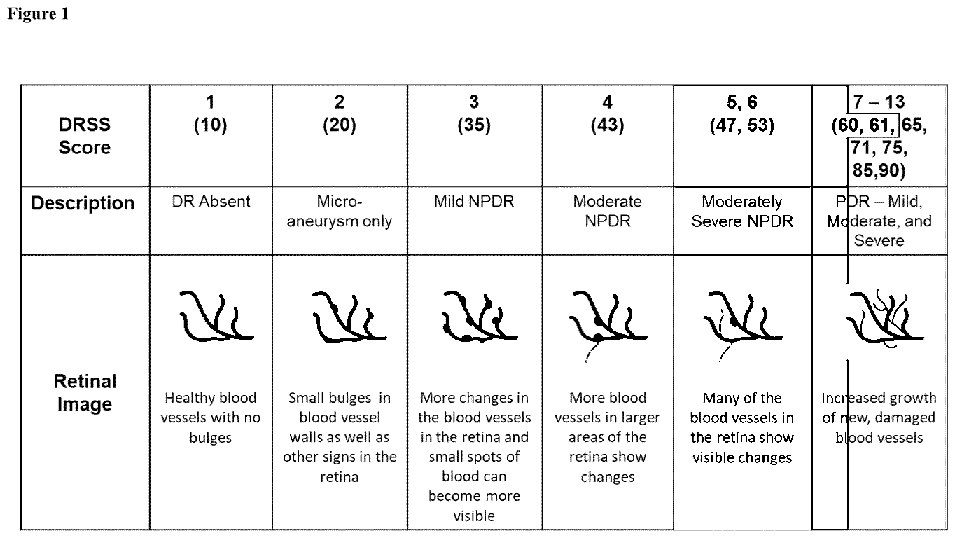

The study is to be configured as a placebo-controlled, double-masked, randomized, Phase 2 study in approximately 100 subjects with moderately severe to severe NPDR (diabetic retinopathy severity score [DRSS] Level 47 or 53, see FIG. 1) or mild PDR (DRSS Level 61), evaluating safety and efficacy following administration of Compound 1 twice daily for 24 weeks. The study will have a 1:1 randomization (placebo: Compound 1). Randomization will be stratified by level of disease severity (NPDR or PDR). Subjects with mild PDR will be capped at 20% for each arm. Efficacy evaluations at 12 and/or 24 weeks will include DRSS, center-involved diabetic macular edema (DME), moderate PDR or PDR-related adverse events (AEs), best-corrected visual acuity (BCVA), and central subfield thickness (CST). Further experimental procedures and results are described below.

### Part I—Experimental Procedures

The total length of subject participation will be approximately 26 weeks, with 5 clinic visits, 4 telephone safety calls, and one telephone call follow-up visit summarized below:
Screening Visit 1 (up to 7 days prior to Baseline Visit)
Qualification/Baseline Visit 2 (1 day)
Treatment-study period (24 weeks)
   Treatment Visit 4 (Week 4), Visit 6 (Week 12), and Visit 9 (Week 24)
   Telephone Safety Call Visit 3 (Week 1), Visit 5 (Week 8), Visit 7 (Week 16), and Visit 8 (Week 20)
Follow-up Phone Call Visit 10 (2 days)

Human subjects will be screened for potential enrollment and, if qualified, enrolled in the study. Inclusion criteria and exclusion criteria for the study are set forth below. Human subjects can qualify in either eye. The eligible eye with the higher DRSS will be designated as the study eye for the primary endpoint efficacy analysis. If the PDR cap is reached, the study eye may be an eye with the lower DRSS, if the other eye has mild PDR. If both eyes have the same DRSS, the eye with the worse BCVA will be selected as the study eye. If the DRSS and BCVA are equivalent between both eyes, the study eye will be the right eye.

### Inclusion Criteria

Males or non-pregnant females ≥18 years of age.
At least one eye with diabetic retinopathy graded at least moderately severe to severe NPDR or mild PDR (corresponding to DRSS 47, 53, or 61, confirmed by a central reading center) in which panretinal laser photocoagulation (PRP) and intravitreal injections of an anti-VEGF agent can be safely deferred for ≥6 months in the opinion of the Investigator.
BCVA assessed by Early Treatment Diabetic Retinopathy Study (ETDRS) protocol letters score of ≥60 letters (Snellen equivalent≥20/63) in the study eye.
Sufficiently clear ocular media, adequate pupillary dilation, and fixation to permit quality fundus imaging in both eyes.
Able to cooperate sufficiently for ophthalmic visual function testing and anatomic assessment.
Body mass index (BMI) between 18 and 40 kg/m², inclusive.
Able and willing to give signed informed consent and follow study instructions.
Able to self-administer oral study medication or to have study medication administered by a caregiver throughout the study period.

### Exclusion Criteria—Ophthalmic

Retinopathy from causes other than diabetes.
Presence of center-involved diabetic macular edema (DME) defined as a central subfield thickness (CST)≥300 μm on SD-OCT or the presence of intra- or subretinal fluid within the central subfield. Center-involved DME in the fellow eye is allowed. Intravitreal injections of an anti-VEGF agent in the fellow eye does not exclude the subject.
Any prior treatment in the study eye with excluded concomitant medication/treatment:
   Focal or grid laser photocoagulation within the past year or PRP at any time.
   Systemic or intravitreal anti-VEGF agents within the last 6 months or likely, in the opinion of the Investigator, to require treatment during the course of the study.
   Intraocular steroids including triamcinolone and dexamethasone implant within the last 6 months.
   Fluocinolone implant within the last 3 years.
Clinically significant ocular disease in either eye as deemed by the Investigator to likely interfere with the study procedures and visual acuity measurements (e.g., cataract, pseudophakia without evidence of posterior capsular opacity, glaucoma, corneal edema, uveitis, severe keratoconjunctivitis sicca).
Presence of other macular or retinal vascular disease including age-related macular degeneration, pattern dystrophy, choroidal neovascularization of any cause, retinal vein occlusion, retinal artery occlusion in the study eye.
Presence of active vitreous hemorrhage that would prevent adequate clinical imaging in either eye.
History of retinal detachment or full-thickness macular hole in the study eye.
Uncontrolled glaucoma in either eye, defined as advanced cup-to-disc ratio>0.7 and intraocular pressure (IOP)>25 mmHg, with or without topical antihypertensive eye drops; treatment of ocular hypertension or controlled glaucoma are not criteria for exclusion.
Ocular incisional surgery including cataract surgery in the study eye within 3 months prior to Day 1.
Yttrium aluminum garnet (YAG) posterior capsulotomy in the study eye within the last 30 days.
Aphakia in the study eye.
Previous pars plana vitrectomy in the study eye.
Epiretinal membrane, posterior hyaloidal traction, and/or vitreomacular traction in the study eye as determined to be significant by the Investigator.
Active uveitis and/or vitritis in either eye.
History of idiopathic or autoimmune-associated uveitis in either eye.
Active infection in either eye including infectious conjunctivitis, keratitis, scleritis, or endophthalmitis.

### Exclusion Criteria—Systemic

Poorly controlled diabetes, defined as hemoglobin A1c (HbA1c)≥12.0% or <12.0% with uncontrolled diabetes mellitus or no HbA1c available.
Known to be immunocompromised or receiving immunosuppressive therapy.
Any disease or medical condition that in the opinion of the Investigator would prevent the subject from successfully participating in the study or which might confound the study results.
Clinically significant systemic disease (e.g., uncontrolled diabetes, myasthenia gravis, cancer, hepatic, renal, endocrine, or cardiovascular disorders) that might interfere with the study as deemed by the Investigator.
Estimated glomerular filtration rate (eGFR)<30 mL/min by Modification of Diet or Renal Disease (MDRD) or creatinine>4 mg/dL.
History of allergic reaction to investigational drug or any of its components.
Resting heart rate (HR) outside the specified range of 50-110 beats per minute at the Screening Visit. HR may be repeated only once if outside the specified range following at least a 5-minute rest period in the sitting position.
Hypertension with resting diastolic blood pressure (BP)>110 mmHg or systolic BP>180 mmHg at the Screening Visit. BP may be repeated only once if outside the specified range following at least a 5-minute rest period in the sitting position.
History of chronic liver disease or presence of elevated alanine aminotransferase (ALT) and aspartate aminotransferase (AST) consistent with such diagnosis (i.e., AST or ALT>2×upper limit of normal).
Participation in any investigational study within 30 days prior to Screening or planning to participate in any other investigational drug or device clinical trials within 30 days of study completion.
Women of childbearing potential who are pregnant, nursing, planning a pregnancy, or not using a medically acceptable form of birth control. All women of childbearing potential must have a negative urine pregnancy test result at the Visit 1/Screening examination and must intend to not become pregnant during the study.

Patients with DME in the fellow eye will be eligible for enrollment into the study, however center-involved DME in the study eye is exclusionary.

Subjects will be screened at Visit 1 and those successfully completing eligibility requirements will return to site for their Qualification/Baseline Visit (Visit 2/Day 1) where they will undergo a set of safety and lab test assessments and study medication will be dispensed. Subjects will then return to site at Visit 4 (Week 4), Visit 6 (Week 12) and Visit 9 (Week 24) for safety and efficacy assessments. In between these site visits, subjects will be contacted by telephone on Visit 3 (Week 1), Visit 5 (Week 8), Visit 7 (Week 16), and Visit 8 (Week 20) for a safety assessment to include AEs, concomitant medications, and drug accountability.

Study medication will be dispensed initially at Visit 2 (Day 1) and then at Visit 4 (Week 4) and Visit 6 (Week 12) at the site. Subjects will bring all unused study medication to each site visit for drug accountability. Study medication will be collected at site during Visit 9 (Week 24). A Follow-up Phone Call will be conducted one week after Visit 9 (Week 24) for AE and concomitant medication assessments.

At the Screening (Visit 1, Day −7 to −1):
A member of the study center staff will interview the individual as to their qualifications for participation in the study, and if the subject wishes to continue, the informed consent form will be signed.
The start of Screening includes the assignment of a subject identification number, an explanation of the study, a medical and ophthalmic history, demographics, and a review of prior/concomitant medications.
Then the following will be conducted: physical examination, measurement of HR/BP, and a urine pregnancy test for women of childbearing potential.
Subsequently, BCVA will be measured and SD-OCT (for CST) and color fundus photographs (for DRSS) will be performed. DRSS eligibility will be determined by a central reading center with 7-field or 4-wide field fundus photography. The central reading center will also determine CST eligibility with SD-OCT. BCVA (distance) can be measured with a Standard ETDRS illuminated chart (on wall or stand) at 4 m.

Then the following will be conducted: assessments of blood chemistry and hematology, ophthalmic examination that includes biomicroscopy and direct or indirect ophthalmoscopy, IOP assessment, eGFR, and AEs.

If the subject meets all eligibility criteria (including DRSS and SD-OCT), then BCVA, DRSS, CST and other safety assessments performed at Screening will be the baseline values, and the subject will be asked to return for their Qualification Visit.

At the Qualification/Baseline (Visit 2, Day 1):
The subject will be randomized into the study.
Study medication will be dispensed in accordance with the subjects randomized treatment arm.
Blood samples will be drawn pre-dose for baseline levels and on Visit 9 (Week 24) for exploratory biomarker processing (i.e., cytokine and Ref-1 levels) to evaluate pharmacodynamic properties.
Subjects will be instructed to administer their study medication (APX3330 or placebo) cach day, with 3 tablets every morning and 2 tablets every evening. Study medication should be taken at approximately the same time each day and may be taken with or without food.

Telephone safety calls will be conducted for Visit 3 (Week 1±2 Days), Visit 5 (Week 8±2 Days), Visit 7 (Week 16±2 Days), and Visit 8 (Week 20±2 Days). The safety assessment will include review of drug compliance, concomitant medications, AEs, and urine pregnancy test at home (only for women of childbearing potential).

At the First Treatment Visit (Visit 4, Week 4±2 Days), subjects will return to the site for the following series of safety and efficacy assessments: drug accountability, concomitant medications, urine pregnancy test (only for women of childbearing potential), HR/BP/vital signs, BCVA (ETDRS), biomicroscopy, ophthalmoscopy, IOP, and AEs. Following the completion of the assessments, used medication kits will be collected for accountability (by counting the number of unused tablets), and new study medication kits will be dispensed.

At the Second Treatment Visit (Visit 6, Week 12±2 Days), subjects will return to the site for the following series of safety and efficacy assessments: drug accountability, concomitant medications, urine pregnancy test (only for women of childbearing potential), HR/BP/vital signs, BCVA (ETDRS), DRSS, CST (SD-OCT), blood draw for PK, blood chemistry, blood hematology, biomicroscopy, ophthalmoscopy, IOP, eGFR, and AEs. Following the completion of the assessments, used medication kits will be collected for accountability (by counting the number of unused tablets), and new study medication kits will be dispensed.

At the Third Treatment Visit (Visit 9, Week 24±2 Days), subjects will return to the site for the following series of safety and efficacy assessments: drug accountability, concomitant medications, urine pregnancy test (only for women of childbearing potential), physical examination, HR/BP/vital signs, BCVA (ETDRS), DRSS, CST (SD-OCT), blood chemistry, blood hematology, biomicroscopy, ophthalmoscopy, IOP, cGFR, AEs, and blood draw for exploratory biomarkers (ELISA, cytokine panel, comprehensive metabolic panel). Visit 9 (Week 24) is the end of the treatment period. Study medication will be returned for accountability (by counting the number of unused tablets), and no further study medication will be dispensed.

A telephone follow-up call will be conducted for Visit 10 (Week 25±2 Days) to evaluate concomitant medications and AEs.

Study subjects will receive study medication as set forth in Table 1 according to the Treatment Group to which the subject is assigned. Subjects will be instructed to take study medication at approximately the same time each day, and the medication may be taken with or without food. Study medication is listed in Table 2.

TABLE 1
Treatment Groups

| Treatment Group | Study Medication and Administration Protocol |
|---|---|
| 1 | Five tablets each containing 120 mg of Compound 1 |
| | by mouth each day, with 3 tablets every morning |
| | and 2 tablets every evening for 24 weeks, for |
| | subjects randomized to active treatment. |
| 2 | Five Placebo tablets by mouth each day, with |
| | 3 tablets every morning and 2 tablets every evening |
| | for 24 weeks, for subjects randomized to placebo. |

TABLE 2
Study Medication

| Study | |
|---|---|
| Medication | Composition of Study Medication |
| 120-mg | The immediate-release tablets consist of a mixture of |
| Tablets of | intragranular components to which an extragranular layer is |
| Compound 1 | applied, all being compressed as circular disks and film- |
| | coated with Opadry Yellow (pale-orange to light-yellow |
| | tablets). |
| | The intragranular components are: |
| | Compound 1 (120 mg per tablet) |
| | Lactose monohydrate |
| | Microcrystalline cellulose NF (Avicel PH101) |
| | Starch 1500 |
| | Sodium carboxymethylcellulose (Aqualon 7MF PH) |
| | Methylcellulose A15LV USP. |
| | The extragranular components are: |
| | Microcrystalline cellulose NF (Avicel PH102) |
| | Sodium carboxymethylcellulose (Aqualon 7MF PH) |
| | Magnesium stearate. |
| Placebo | Placebo tablets are immediate release, identical in shape |
| Tablets | and color to the tablets containing 120 mg of Compound 1, |
| | except for the absence of the active pharmaceutical |
| | ingredient. |

Any subject is permitted to voluntarily withdraw from the study at any time without prejudice. A non-completing subject is defined as one who exited the study by their own volition or at the discretion of the Investigator and/or the Medical Monitor prior to completing all of the study procedures required in the protocol.

If a subject considers discontinuing from the study due to an AE, the Investigator may offer a dose reduction from 600 mg to 480 mg per day as an alternative (2 tablets every morning and 2 tablets every evening).

### Evaluation of Efficacy and Safety—Endpoints and Measurement Procedures

The primary efficacy endpoint will be the percent of subjects with a ≥2-step improvement in DRSS in the study eye at Week 24. Secondary efficacy endpoints will include:
Percent of subjects with an improvement or worsening in DRSS (see FIG. 1) of ≥1, ≥2, ≥3, and ≥4 steps at Week 12 and Week 24,
Mean change from baseline in DRSS at Week 24,
Percent of subjects not developing center-involved DME or moderate PDR or PDR-related AEs during the study at Week 12 and Week 24,
Mean change from baseline in BCVA at Week 24, and
Mean change from baseline in CST.

Primary and secondary endpoints will be evaluated in the study eyes, fellow eyes, all qualified eyes (study eyes and fellow eyes that meet all study eye eligibility criteria), and either eye (i.e., best response). All of the efficacy endpoints will also be analyzed by modified intention-to-treat (mITT) and per protocol (PP) populations. Other subpopulations may be identified and analyzed.

Exploratory efficacy endpoints will include:
Relationship between Compound 1 in plasma measured at Week 12, and change from baseline in DRSS in the study eye at Week 12 and Week 24,
Mean change from baseline in plasma Ref-1 levels at Week 24,
Mean change from baseline in cytokine levels at Week 24,
Relationship between Ref-1 levels in plasma and change from baseline in DRSS in the study eye at Week 24, and
Relationship between cytokines levels in plasma and change from baseline in DRSS in the study eye at Week 24.

Measurements will be determined as follows, where every effort will be made to have the same person perform the measurements at all timepoints and at all visits:
DRSS will be measured with 7-field or 4-wide field color fundus photographs,
CST will be measured using SD-OCT, and
BCVA will be measured by Standard ETDRS chart at 4 m (letters).

For pharmacokinetics analysis, at Visit 6 (Week 12±2 days) pre-morning dose and 3 hours post-morning dose, blood samples will be collected to establish drug levels of Compound 1 from approximately 25 to 30 subjects at a subset of clinical sites. These subjects will be instructed to delay their morning study medication dose on the day of this visit so that they will take their study medication at the site. Five mL of blood will be drawn immediately pre-dosing to establish a steady-state drug level. A second 5-mL sample will be drawn 3 hours later to establish the Cₘₐₓ drug level. Analysis of plasma samples for Compound 1 concentration determinations will be performed by a central PK laboratory using a validated liquid chromatography-mass spectrometry and liquid chromatography-tandem mass spectrometry (LC-MS/MS) method.

Safety endpoints will include:
Incidence and severity of systemic and ocular AEs,
Change from baseline in body system assessments,
Change from baseline in vital sign measurements,
Change from baseline in clinical laboratory assay results (blood chemistry, hematology),
Change from baseline in intraocular pressure (IOP),
Change from baseline in slit-lamp examination parameters,
Change from baseline in dilated funduscopic examination parameters,
Percent of subjects with a decrease of ≥ 10 letters in BCVA compared to baseline at Week 12 and Week 24,
Percent of subjects progressing to center-involved DME (eligible for rescue treatment at Week 12 and Week 24),
Percent of subjects with a worsening in DRSS of >2 steps in the study eye at Week 12 and Week 24,
Percent of subjects developing anterior segment neovascularization at Week 12 and Week 24,
Percent of subjects rescued (intravitreal anti-VEGF injection, laser PRP, focal/grid laser treatment, or surgery [vitrectomy]) at the discretion of the Investigator at Week 12 and Week 24, and
Change from baseline in estimated glomerular filtration rate (eGFR) at Week 12 and Week 24.

### Evaluation of Efficacy and Safety—General Analysis Procedures

Analysis populations will include:
Modified Intention-to-Treat (mITT): The mITT will include all randomized subjects who received at least one dose of study treatment and at least one post-dose efficacy measurement. The mITT will be used to analyze efficacy endpoints.
Per Protocol Population (PP): The PP population will include all subjects in the mITT who have missed less than 20% of expected doses and do not have any major protocol deviations considered to have significant impact on treatment outcome. The PP population will be used for primary endpoint analysis and to analyze efficacy endpoints.
All Randomized Population (ARP): The ARP will include all randomized subjects. This population is also known as the Intent-to-Treat (ITT) population. The ARP will be used in confirmatory efficacy analyses.
Safety Population (SP): The SP will include all randomized subjects who have received at least one dose of study treatment. The SP will be used to summarize safety variables.

### Part II—Results

Provided below is data on safety on Compound 1 orally administered to patients according to the study protocol described herein. The data is from one-hundred patients that were enrolled in the trial. Per the study protocol described above, patients enrolled in the study were randomized 1:1 for receipt of placebo versus Compound 1. The safety data below is the combined results from patients that received placebo and those received Compound 1. The results show that Compound 1 administered according to the study protocol had a good safety profile in the patient population enrolled in this study.

Table 3 below provides results from analysis of concentration of alanine aminotransferase in subjects' blood.

TABLE 3

| | Number of Subjects for | |
|---|---|---|
| | Which Have Data for Mean | Mean Concentration |
| | Concentration of Alanine | of Alanine |
| Time | Aminotransferase in | Aminotransferase in |
| Point | Subjects' Blood | Subjects' Blood (IU/L) |
| Baseline | 97 | 22.4 |
| Week 12 | 53 | 21.6 |
| Week 24 | 24 | 20.4 |

Comparison of data available for 50 subjects at week 12 showed a change of −0.8 IU/L in mean concentration of alanine aminotransferase in subjects' blood relative to the mean concentration of alanine aminotransferase in subjects' blood at Baseline. This corresponds to a 3.6% reduction in mean concentration of alanine aminotransferase in subjects' blood at week 12 relative to the mean concentration of alanine aminotransferase in subjects' blood at Baseline.

Comparison of data available for 22 subjects at week 24 showed a change of −2.1 IU/L in mean concentration of alanine aminotransferase in subjects' blood relative to the mean concentration of alanine aminotransferase in subjects' blood at Baseline. This corresponds to a 9.4% reduction in mean concentration of alanine aminotransferase in subjects' blood at week 24 relative to the mean concentration of alanine aminotransferase in subjects' blood at Baseline.

Table 4 below provides results from analysis of concentration of aspartate aminotransferase in subjects' blood.

TABLE 4

| | Number of Subjects for | |
|---|---|---|
| | Which Have Data for Mean | Mean Concentration |
| | Concentration of Aspartate | of Aspartate |
| Time | Aminotransferase in | Aminotransferase in |
| Point | Subjects' Blood | Subjects' Blood (IU/L) |
| Baseline | 97 | 19.7 |
| Week 12 | 53 | 19.2 |
| Week 24 | 24 | 17.5 |

Comparison of data available for 50 subjects at week 12 showed a change of −0.5 IU/L in mean concentration of aspartate aminotransferase in subjects' blood relative to the mean concentration of aspartate aminotransferase in subjects' blood at Baseline. This corresponds to a 3% reduction in mean concentration of aspartate aminotransferase in subjects' blood at week 12 relative to the mean concentration of aspartate aminotransferase in subjects' blood at Baseline.

Comparison of data available for 22 subjects at week 24 showed a change of −1.8 IU/L in mean concentration of aspartate aminotransferase in subjects' blood relative to the mean concentration of aspartate aminotransferase in subjects' blood at Baseline. This corresponds to a 9% reduction in mean concentration of aspartate aminotransferase in subjects' blood at week 24 relative to the mean concentration of aspartate aminotransferase in subjects' blood at Baseline.

Table 5 below provides results from analysis of glomerular filtration rate of subjects.

TABLE 5

| | Number of Subjects for | |
|---|---|---|
| Time | Which Have Data for Mean | Mean Glomerular Filtration |
| Point | Glomerular Filtration Rate | Rate (mL/min/1.73 m²) |
| Baseline | 94 | 92.53 |
| Week 12 | 52 | 83.85 |
| Week 24 | 24 | 77.7 |

Comparison of data available for 47 subjects at week 12 showed a change of −2.24 mL/min/1.73 m² in mean glomerular filtrate rate in subjects relative to the mean glomerular filtrate rate in subjects at Baseline. This corresponds to a 2% reduction in mean glomerular filtrate rate in subjects at week 12 relative to the mean glomerular filtrate rate in subjects at Baseline.

Comparison of data available for 21 subjects at week 24 showed a change of −9.84 mL/min/1.73 m² in mean glomerular filtrate rate in subjects relative to the mean glomerular filtrate rate in subjects at Baseline. This corresponds to a 11% reduction in mean glomerular filtrate rate in subjects at week 24 relative to the mean glomerular filtrate rate in subjects at Baseline.

Table 6 below provides results from analysis of heart rate of subjects.

TABLE 6

| Time | Number of Subjects for Which | Mean Heart Rate |
|---|---|---|
| Point | Have Data for Mean Heart | (beats/min) |
| Baseline | 99 | 76.7 |
| Week 12 | 62 | 76.3 |
| Week 24 | 27 | 80.7 |

Comparison of data available for 62 subjects at week 12 showed a change of +0.9 beats/min in mean heart rate in subjects relative to the mean heart rate in subjects at Baseline. This corresponds to a 1% increase in mean heart rate in subjects at week 12 relative to the mean heart rate in subjects at Baseline.

Comparison of data available for 27 subjects at week 24 showed a change of +4.3 beats/min in mean heart rate in subjects relative to the mean heart rate in subjects at Baseline. This corresponds to a 6% increase in mean heart rate in subjects at week 24 relative to the mean heart rate in subjects at Baseline.

Table 7 below provides results from analysis of systolic blood pressure of subjects.

TABLE 7

| | Number of Subjects for Which | |
|---|---|---|
| Time | Have Data for Mean Systolic | Mean Systolic Blood |
| Point | Blood Pressure | Pressure (mmHg) |
| Baseline | 99 | 136.9 |
| Week 12 | 62 | 132 |
| Week 24 | 27 | 139.7 |

Comparison of data available for 62 subjects at week 12 showed a change of −4.7 mmHg in mean systolic blood pressure in subjects relative to the mean systolic blood pressure in subjects at Baseline. This corresponds to a 3% reduction in mean systolic blood pressure in subjects at week 12 relative to the mean systolic blood pressure in subjects at Baseline.

Comparison of data available for 27 subjects at week 24 showed a change of −0.7 mmHg in mean systolic blood pressure in subjects relative to the mean systolic blood pressure in subjects at Baseline. This corresponds to a 1% reduction in mean systolic blood pressure in subjects at week 24 relative to the mean systolic blood pressure in subjects at Baseline.

Table 8 below provides results from analysis of diastolic blood pressure of subjects.

TABLE 8

| | Number of Subjects for Which | |
|---|---|---|
| Time | Have Data for Mean Diastolic | Mean Diastolic Blood |
| Point | Blood Pressure | Pressure (mmHg) |
| Baseline | 99 | 80.3 |
| Week 12 | 62 | 77.1 |
| Week 24 | 27 | 80.4 |

Comparison of data available for 62 subjects at week 12 showed a change of −2.8 mmHg in mean diastolic blood pressure in subjects relative to the mean diastolic blood pressure in subjects at Baseline. This corresponds to a 3% reduction in mean diastolic blood pressure in subjects at week 12 relative to the mean diastolic blood pressure in subjects at Baseline.

Comparison of data available for 27 subjects at week 24 showed a change of +1 mmHg in mean diastolic blood pressure in subjects relative to the mean diastolic blood pressure in subjects at Baseline. This corresponds to a 1% increase in mean diastolic blood pressure in subjects at week 24 relative to the mean diastolic blood pressure in subjects at Baseline.

Table 9 below provides a summary of occurrence of adverse events across 100 subjects enrolled in the study, which is the combined results from patients that received placebo and those that received Compound 1. Of these 100 subjects, 41 subjects reported at least one treatment-emergent adverse event. A total of 83 treatment-emergent adverse events were observed from the 100 subject enrolled in the study. A subject reporting more than one treatment-emergent adverse event is only counted once within the System Organ Class in Table 9.

TABLE 9

| | No. of Subjects That |
|---|---|
| | Experienced a |
| | Treatment-Emergent |
| Category of Adverse Event | Adverse the Category |
| Eye disorder | 9 |
| Anterior^{[1]} | 5 |
| Posterior^{[1]} | 9 |
| Gastrointestinal disorder | 9 |
| Infection or infestation | 9 |
| Nervous system disorder | 7 |
| Skin and subcutaneous tissue disorder | 7 |
| Musculoskeletal and connective tissue disorder | 5 |
| Not coded | 4 |
| Investigation | 3 |
| Psychiatric disorder | 2 |
| Respiratory, thoracic, or mediastinal disorder | 2 |
| Blood or lymphatic system disorder | 1 |
| Cardiac disorder | 1 |
| Ear or labyrinth disorder | 1 |
| General disorders or administration site | 1 |
| condition | |
| Hepatobiliary disorder | 1 |
| Injury, poisoning, or procedural complication | 1 |
| Metabolism or nutrition disorder | 1 |
| Reproductive system or breast disorder | 1 |
| Vascular disorder | 1 |

^{[1]}Bilateral ocular events are counted twice, i.e., once for each eye.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a diabetic retinal disease in a human patient, comprising orally administering to a human patient in need thereof a first therapeutic agent in an amount of from about 480 mg to about 600 mg per day, to thereby treat the diabetic retinal disease, wherein the first therapeutic agent is a compound of Formula I or a pharmaceutically acceptable salt thereof:

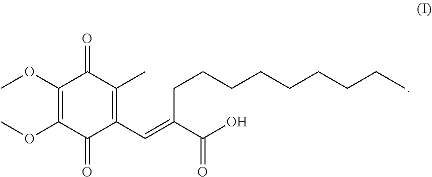 [SMILES: CCCCCCCCC/C(=C\C1=C(C)C(=O)C(OC)=C(OC)C1=O)C(=O)O]

2. The method of claim 1, wherein the first therapeutic agent is a compound of Formula I.

3. The method of claim 1 or 2, wherein a first dose of the first therapeutic agent and a second dose of the first therapeutic agent are orally administered to the patient on the same day.

4. The method of any one of claims 1-3, wherein the first therapeutic agent is orally administered to the patient in an amount of about 600 mg per day.

5. The method of claim 1 or 2, wherein about 360 mg of the first therapeutic agent is orally administered to the patient in the morning, and about 240 mg of the first therapeutic agent is orally administered to the patient in the evening.

6. The method of claim 1 or 2, wherein about 360 mg of the first therapeutic agent is orally administered to the patient, and then at a time that is from about 8 hours to about 16 hours later about 240 mg of the first therapeutic agent is orally administered to the patient.

7. The method of claim 1 or 2, wherein about 240 mg of the first therapeutic agent is orally administered to the patient in the morning, and about 360 mg of the first therapeutic agent is orally administered to the patient in the evening.

8. The method of claim 1 or 2, wherein about 240 mg of the first therapeutic agent is orally administered to the patient, and then at a time that is from about 8 hours to about 16 hours later about 360 mg of the first therapeutic agent is orally administered to the patient.

9. The method of claim 1 or 2, wherein about 300 mg of the first therapeutic agent is orally administered to the patient in the morning, and about 300 mg of the first therapeutic agent is orally administered to the patient in the evening.

10. The method of claim 1 or 2, wherein about 300 mg of the first therapeutic agent is orally administered to the patient, and then at a time that is from about 8 hours to about 16 hours later about 300 mg of the first therapeutic agent is orally administered to the patient.

11. The method of any one of claims 1-10, wherein if the patient experiences an adverse event due to the first therapeutic agent, then thereafter for a period of at least two days the first therapeutic agent is orally administered to the patient in the reduced-daily amount of about 480 mg per day.

12. The method of any one of claims 1-3, wherein the first therapeutic agent is orally administered to a patient in an amount of about 480 mg per day.

13. The method of claim 11 or 12, wherein about 240 mg of the first therapeutic agent is orally administered to the patient in the morning, and about 240 mg of the first therapeutic agent is orally administered to the patient in the evening.

14. The method of claim 11 or 12, wherein about 240 mg of the first therapeutic agent is orally administered to the patient, and then at a time that is from about 8 hours to about 16 hours later about 240 mg of the first therapeutic agent is orally administered to the patient.

15. The method of any one of claims 1-10, wherein if the patient experiences an adverse event due to the first therapeutic agent, then thereafter for a period of at least two days the first therapeutic agent is orally administered to the patient in the reduced-daily amount of about 300 mg per day.

16. The method of claim 15, wherein the first therapeutic agent is orally administered to the patient in the morning.

17. The method of claim 1 or 2, wherein the first therapeutic agent is orally administered to a patient only 1 time per day.

18. A method of treating a diabetic retinal disease in a human patient, comprising orally administering to a human patient in need thereof a first therapeutic agent in an amount of from about 120 mg to about 600 mg per day, to thereby treat the diabetic retinal disease, wherein the first therapeutic agent is a compound of Formula I or a pharmaceutically acceptable salt thereof:

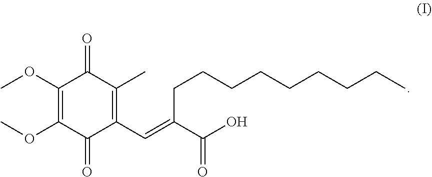 [SMILES: CCCCCCCCC/C(=C\C1=C(C)C(=O)C(OC)=C(OC)C1=O)C(=O)O]

19. The method of claim 18, wherein the first therapeutic agent is a compound of Formula I.

20. The method of claim 18 or 19, wherein the first therapeutic agent is orally administered to the patient in an amount of about 300 mg per day.

21. The method of claim 18 or 19, wherein the first therapeutic agent is orally administered to the patient in an amount of about 240 mg per day.

22. The method of claim 18 or 19, wherein the first therapeutic agent is orally administered to the patient in an amount of about 120 mg per day.

23. The method of any one of claims 18-22, wherein a first dose of the first therapeutic agent and a second dose of the first therapeutic agent are orally administered to the patient on the same day.

24. The method of any one of claims 18-23, wherein the first therapeutic agent is orally administered to the patient in the morning.

25. The method of any one of claims 18-24, wherein the first therapeutic agent is orally administered to the patient in the evening.

26. The method of any one of claims 18-22, wherein the first therapeutic agent is orally administered to the patient only 1 time per day.

27. The method of any one of claims 1-26, wherein the amount of the first therapeutic agent is orally administered to the patient daily for at least 4 weeks.

28. The method of any one of claims 1-26, wherein the amount of the first therapeutic agent is orally administered to the patient daily for at least 12 weeks.

29. The method of any one of claims 1-26, wherein the amount of the first therapeutic agent is orally administered to the patient daily for at least 24 weeks.

30. The method of any one of claims 1-29, wherein the first therapeutic agent is orally administered to the patient in the form of an extended-release pharmaceutical composition.

31. The method of any one of claims 1-30, further comprising administering to the patient a second therapeutic agent that is an anti-inflammatory agent, anti-angiogenic agent, tyrosine kinase inhibitor, angiopoietin-2 inhibitor, and/or vascular endothelial growth factor inhibitor.

32. The method of any one of claims 1-30, further comprising administering to the patient a second therapeutic agent that is a vascular endothelial growth factor inhibitor.

33. The method of any one of claims 1-32, wherein the diabetic retinal disease is diabetic retinopathy.

34. The method of claim 33, wherein the diabetic retinopathy is mild diabetic retinopathy.

35. The method of claim 33, wherein the diabetic retinopathy is moderate diabetic retinopathy.

36. The method of claim 33, wherein the diabetic retinopathy is moderately severe to severe diabetic retinopathy.

37. The method of any one of claims 33-36, wherein the diabetic retinopathy is non-proliferative diabetic retinopathy.

38. The method of any one of claims 33-36, wherein the diabetic retinopathy is proliferative diabetic retinopathy.

39. The method of any one of claims 1-32, wherein the diabetic retinal disease is diabetic macular edema.

40. The method of any one of claims 1-39, wherein the human patient is an adult human patient.

41. The method of any one of claims 1-39, wherein the method reduces a symptom of diabetes.

42. A method of treating a disease or condition selected from wet age-related macular degeneration, dry age-related macular degeneration, retinal vein occlusion, geographic atrophy, retinal neovascularization, choroidal neovascularization, or corneal graft rejection, comprising orally administering to a human patient in need thereof a first therapeutic agent in an amount of from about 120 mg to about 600 mg per day, to thereby treat the disease or condition, wherein the first therapeutic agent is a compound of Formula I or a pharmaceutically acceptable salt thereof:

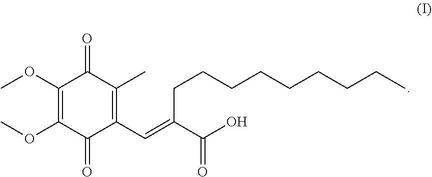 [SMILES: CCCCCCCCC/C(=C\C1=C(C)C(=O)C(OC)=C(OC)C1=O)C(=O)O]

43. The method of claim 42, wherein the first therapeutic agent is a compound of Formula I.

44. The method of claim 42 or 43, wherein the first therapeutic agent is orally administered to the patient in an amount of about 600 mg per day.

45. The method of claim 42 or 43, wherein the first therapeutic agent is orally administered to the patient in an amount of about 480 mg per day.

46. The method of claim 42 or 43, wherein the first therapeutic agent is orally administered to the patient in an amount of about 300 mg per day.

47. The method of claim 42 or 43, wherein the first therapeutic agent is orally administered to the patient in an amount of about 240 mg per day.

48. The method of claim 42 or 43, wherein the first therapeutic agent is orally administered to the patient in an amount of about 120 mg per day.

49. The method of any one of claims 42-48, wherein a first dose of the first therapeutic agent and a second dose of the first therapeutic agent are orally administered to the patient on the same day.

50. The method of any one of claims 42-49, wherein the first therapeutic agent is orally administered to the patient in the morning.

51. The method of any one of claims 42-50, wherein the first therapeutic agent is orally administered to the patient in the evening.

52. The method of any one of claims 42-48, wherein the first therapeutic agent is orally administered to the patient only 1 time per day.

53. The method of any one of claims 42-52, further comprising administering to the patient a second therapeutic agent that is an anti-inflammatory agent, anti-angiogenic agent, tyrosine kinase inhibitor, angiopoietin-2 inhibitor, and/or vascular endothelial growth factor inhibitor.

54. The method of any one of claims 1-53, wherein the method reduces any renal impairment experienced by the patient.

55. The method of any one of claims 1-54, wherein the method achieves a neuroprotective effect.

56. The method of any one of claims 1-55, wherein any increase in blood plasma concentration of alanine aminotransferase due to the first therapeutic agent is no greater than 5%.

57. The method of any one of claims 1-56, wherein any increase in blood plasma concentration of aspartate aminotransferase due to the first therapeutic agent is no greater than 5%.

58. The method of any one of claims 1-57 wherein any reduction in glomerular filtration rate in the patient is no greater than 15%.

59. The method of any one of claims 1-58, wherein the incidence of any eye disorder due to the first therapeutic agent occurs no more frequently than one patient for every ten patients subjected to the same treatment.

60. The method of any one of claims 1-59, wherein the incidence of any eye disorder due to the first therapeutic agent occurs no more frequently than one patient for every twenty patients subjected to the same treatment.

61. The method of any one of claims 1-60, wherein the incidence of any gastrointestinal disorder due to the first therapeutic agent occurs no more frequently than one patient for every ten patients subjected to the same treatment.

62. The method of any one of claims 1-61, wherein the incidence of any nervous system disorder due to the first therapeutic agent occurs no more frequently than one patient for every twenty patients subjected to the same treatment.

63. The method of any one of claims 1-62, further characterized by achieving a reduction in blood plasma concentration of alanine aminotransferase due to the first therapeutic agent.

64. The method of any one of claims 1-63, further characterized by achieving a reduction in blood plasma concentration of aspartate aminotransferase due to the first therapeutic agent.
